(19) United States
(12) Patent Application Publication
(10) Pub. No.: US 2023/0032664 A1
(43) Pub. Date: Feb. 2, 2023

(54) Iron-reducing Tessaraccocus Oleiagri Strain DH10 and Applications thereof

(71) Applicant: YANGTZE UNIVERSITY, Jingzhou City (CN)

(72) Inventors: Yuehui She, Jingzhou City (CN); Hao Dong, Jingzhou City (CN); Shanshan Sun, Jingzhou City (CN); Fan Zhang, Jingzhou City (CN); SanBao Su, Jingzhou City (CN); Han Zhang, Jingzhou City (CN); Xue Weng, Jingzhou City (CN); GaoMing Yu, Jingzhou City (CN); AnYing Zheng, Jingzhou City (CN); Yang Li, Jingzhou City (CN)

(21) Appl. No.: 17/554,463

(22) Filed: Dec. 17, 2021

(30) Foreign Application Priority Data
Jul. 15, 2021 (CN) 2021108026014

(51) Int. Cl. C12N 1/20; C09K 8/582

(52) U.S. Cl. CPC C12N 1/205; C09K 8/582

(57) ABSTRACT

An iron-reducing Tessaracoccus oleiagri strain DH10 and applications of the iron-reducing Tessaracoccus oleiagri strain DH10 are provided. The Tessaracoccus oleiagri strain DH10 had been preserved in China center for type culture collection on Apr. 19, 2021, with a preservation number of CCTCC No: M 2021404. The iron-reducing Tessaracoccus oleiagri strain DH10 can efficiently reduce Fe(III) is separated and screened from oil reservoir environment. SEM analysis shows that it can effectively decompose lean iron montmorillonite minerals and inhibit expansion of clay. Moreover, core experiments show that biological agent of the Tessaracoccus oleiagri strain DH10 can reduce water sensitivity of reservoir core and water injection pressure, and can be applied to crude oil recovery to effectively improve crude oil recovery factor.

### FIELD OF DISCLOSURE

The disclosure relates to the field of microbial enhanced oil recovery (MEOR), and more particularly to an iron-reducing Tessaracoccus oleiagri strain DH10 and applications of the iron-reducing Tessaracoccus oleiagri strain DH10.

### BACKGROUND OF DISCLOSURE

With the development of petroleum industry, there are fewer and fewer conventional oil and gas resources in the country, while social demands for oil and gas resources are becoming higher and higher. This contradiction leads to the current high external dependence of national oil supply, and the energy security is threatened. Oil and gas resources such as low permeability reservoir and water sensitive reservoir will be breakthrough points to increase oil production for a long time, and will also be key research fields. It is common knowledge that water sensitive clay minerals are one of the main factors restricting the development level of low-permeability and water sensitive reservoirs, so stabilizing water sensitive clay minerals is a key point to improve the development level of low-permeability and water sensitive reservoirs. Low permeability and water sensitive reservoirs usually have high clay mineral content. The clay mineral content determines the reservoir permeability. Even a small change in the clay mineral content will cause a great change in the reservoir permeability. When the clay mineral content reaches 35%-40%, the reservoir is almost impermeable. The higher the clay mineral content, the higher the expansion rate of clay and the stronger the cation exchange capacity, the stronger the reservoir water sensitivity, and the type of clay minerals will affect the seepage characteristics of the reservoir. Expansion of clay minerals is a key factor affecting the permeability of low permeability reservoir, and the expansion of clay minerals will reduce the permeability.

Microorganisms with Fe (III) reduction function usually refer to the functional microorganisms that can dissimilatively reduce Fe(III) to Fe(II), which are a special microbial population that can use organic matter as an electron donor. While oxidizing organic matter, Fe³⁺ is an only electron acceptor to reduce Fe(III) to Fe(II), and obtain the energy required for their own growth in the process of reduction and metabolism. The metabolites of iron-reducing microorganisms can change the physical and chemical environment on the mineral surfaces, which is the main driving force of mineral decomposition. The rate of microbial decomposition of minerals is several orders of magnitude higher than that of single chemical decomposition. For example, a study published by Jinwook Kim et al, “Role of Microbes in the Smectite-to-Illite Reaction”, Science, 2004, pp. 830-832, Vol. 303 found that that a strain of dissimilation iron-reducing bacteria Shewanella oneidensis MR-1 can reduce ferric iron in iron-rich montmorillonite within two weeks and promote the transformation of montmorillonite to illite. Daniel R. Bond et al., “Reduction of Fe(III) oxide by methanogens in the presence and absence of extracellular quinones”, Environmental Microbiology, 2002, pp. 115-124, Vol. 4, No. 2 first found that thermophilic anaerobic methanogens can also reduce ferric iron in the structure of clay minerals, resulting in a phase transformation of clay minerals. These findings break a long-standing understanding that the illite transformation process of montmorillonite is controlled by temperature, pressure and time, and break through the limitation of large time scale of transformation between clay minerals. Therefore, it is of great significance to improve crude oil recovery factor by using reservoir iron-reducing microorganisms to inhibit clay hydration expansion or shrinkage expansion and improve seepage environment of crude oil fluid.

In the process of microbial oil recovery, different reservoirs also have certain requirements for strains. There are many iron-reducing bacteria reported so far, but there are very few iron-reducing bacteria separated from oil reservoirs, and functional characteristics of the microorganism Tessaracoccus oleiagri of the genus Tessaracoccus separated from the oil reservoir environment and its application in oil exploitation and suppression of expansion of clay minerals have not been seen related research reports.

### SUMMARY OF DISCLOSURE

In an aspect, an objective of the disclosure is to provide an iron-reducing Tessaracoccus oleiagri strain DH10, which is separated from the oil reservoir environment and has been preserved in China center for type culture collection on Apr. 19, 2021. The address is Wuhan University, Wuhan City, China. The preservation number of the Tessaracoccus oleiagri strain DH10 is CCTCC No: M 2021404.

In another aspect, the disclosure is to provide a microbial agent including the iron-reducing Tessaracoccus oleiagri strain DH10.

In an embodiment, the microbial agent is a solid preparation or a liquid preparation.

In still another aspect, the disclosure is to provide a biological expansion-shrinking bacteria agent, including a nutrient medium; the iron-reducing strain Tessaracoccus oleiagri DH10 or the microbial agent.

In an embodiment, the nutrient medium in the biological expansion-shrinking bacteria agent is a LB medium; or the nutrient medium is a culture medium with a range of pH value of 5.0-9.0 containing 10-50 g/L of sucrose, 10-40 g/L of sodium acetate, 3-20 g/L of sodium lactate, 0.1-2 g/L of MgSO₄, 2-18 g/L of KCl, 5-9 g/L of MnSO₄, 5-10 g/L of CuSO₄, 5-12 g/L of ZnSO₄, 1-7 g/L of KH₂PO₄, and 2-10 g/L of montmorillonite.

In an embodiment, the biological expansion-shrinking bacteria agent is prepared by inoculating the iron-reducing Tessaracoccus oleiagri strain DH10 or the microbial agent into the nutrient medium and then fermenting at pH 5-9.5 and temperature 20-60° C.

In an embodiment, the iron-reducing Tessaracoccus oleiagri strain DH10, the microbial agent or the biological expansion-shrinking bacteria agent is applied to reduction of Fe(III).

In an embodiment, the iron-reducing Tessaracoccus oleiagri strain DH10, the microbial agent or the biological expansion-shrinking bacteria agent is applied to expansion inhibiting of clay.

In an embodiment, the iron-reducing Tessaracoccus oleiagri strain DH10, the microbial agent or the biological expansion-shrinking bacteria agent is applied to reducing of injection pressure of low permeability reservoir.

In an embodiment, the iron-reducing Tessaracoccus oleiagri strain DH10, the microbial agent or the biological expansion-shrinking bacteria agent is applied to enhancement of crude oil recovery factor.

Compared with the prior art, the embodiments of the disclosure mainly have the following beneficial effects:

the disclosure separated and screened the iron-reducing Tessaracoccus oleiagri strain DH10 that can reduce Fe³⁺ from the oil reservoir environment. The analysis of scanning electron microscope and X-ray diffractometer (XRD) shows that the Tessaracoccus oleiagri strain DH10 can effectively decompose and corrode montmorillonite minerals, reduce Fe³⁺ to Fe′, and effectively inhibit expansion of clay. Moreover, core experiments show that biological agent of the Tessaracoccus oleiagri strain DH10 can reduce the water injection pressure in oil development. The application of the Tessaracoccus oleiagri strain DH10 to crude oil recovery can effectively improve crude oil recovery factor.

### BRIEF DESCRIPTION OF DRAWINGS

FIG. 1 shows a color change of culture medium before and after inoculation of a Tessaracoccus oleiagri strain DH10 into Fe(OH)₃ according to embodiment 1 of the disclosure.

FIG. 2 shows a change of Fe³⁺ concentration in montmorillonite with time after inoculation of the Tessaracoccus oleiagri strain DH10 comparing with a blank control group according to embodiment 2 of the disclosure.

FIG. 3A is a scanning electron microscope view of an original montmorillonite sample without an action of the Tessaracoccus oleiagri strain DH10 according to the embodiment 2 of the disclosure.

FIG. 3B is a scanning electron microscope view of a montmorillonite sample after the action of the Tessaracoccus oleiagri strain DH10 for 7 days according to the embodiment 2 of the disclosure.

### DETAILED DESCRIPTION OF EMBODIMENTS

The technical solution of the disclosure will be clearly and completely described below in combination with the illustrated embodiments of the disclosure. Obviously, the illustrated embodiments are only part of the embodiments of the disclosure, not all of the embodiments of the disclosure. Based on the illustrated embodiments of the disclosure, all other embodiments obtained by those skilled in the related art without creative labor belong to the protection scope of the disclosure.

### Embodiment 1 Separation and Identification of Iron-Reducing Bacteria

1. Separation of Iron-Reducing Bacteria

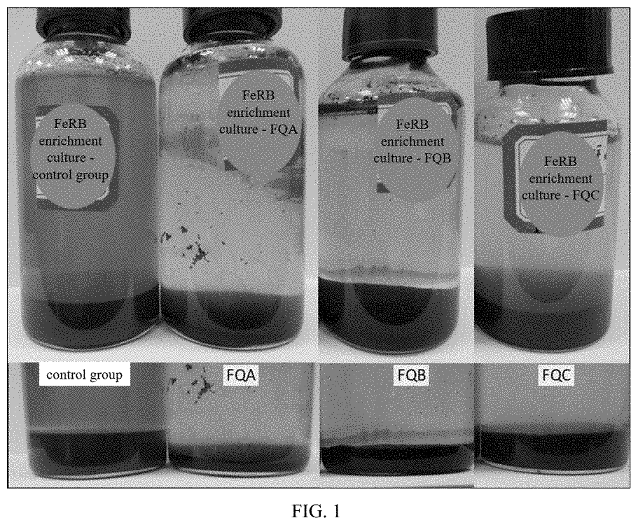

The disclosure provides a strain of iron-reducing bacteria separated from oil reservoir environment. A screening method is as follows. According to the conventional strain screening method, 10 milliliters (ml) of water sample collected from the oil field is taken and put into an anaerobic bottle containing 100 ml of Fe(OH)₃ (0.5% Fe(OH)₃, w: v), then stood for anaerobic culture for one week at 35° C., and an experimental group with obvious solid color darkening is selected. In an anaerobic workstation, 100 microliters (μL) of fermentation broth are taken and coated on a lysogeny broth (LB) agar plate medium under an anaerobic condition, and anaerobic cultured at 35° C. for 48 hours. Single colonies of different morphologies are selected, streaked and purified on the LB agar plate medium, and cultured at 35° C. for 48 hours. The selected single colonies are inoculated into a Fe(OH)₃ medium after enrichment culture, stood for anaerobic culture for 72 hours at 35° C., and strains corresponding to the experimental group with significantly darker solid phase color are selected. In the illustrated embodiment, through preliminary screening, it is found that there are microorganisms in the sample that can significantly darken the color of the solid phase (a color change of the solid phase before and after bacterial culture is shown in FIG. 1), indicating that the screened microorganisms can reduce Fe(III).

In the illustrated embodiment, through preliminary screening of microorganisms that can metabolize with Fe(III) as an only electron receptor, and further cultivation and domestication, a strain capable of reducing Fe(III) is separated and named DH10. The concentrations of Fe(III) and Fe(II) in the sample are determined by spectrophotometry. The results showed that the reduction efficiency of strain DH10 to Fe(III) in the culture medium could reach 35.3% within 4 days.

2. Molecular Biological Identification

According to the 16S rRNA identification method of conventional strain, the genomic DNA of the strain DH10 is extracted, and the corresponding primers are designed for polymerase chain reaction (PCR) amplification to obtain amplified products. The amplified products are detected by agarose gel electrophoresis, and the amplified products are sent to Nanjing Personalbio Gene Technology Co., Ltd. for DNA sequencing to thereby obtain sequencing results. The sequencing results of the PCR amplified products of the strain DH10 are submitted to national center for biotechnology information (NCBI) for search and homology comparison by basic local alignment search tool (BLAST). It is determined that the strain DH10 is Tessaracoccus oleiagri (the similarity with Tessaracoccus oleiagriola strain SL014B-20A11 is 99.72%). The comparison results of 16S rRNA sequence of the strain DH10 and the sequence of the GenBank database are shown in Table 1.

TABLE 1
comparison results between 16S rRNA sequence of
the strain DH10 and sequence of GenBank database

| | Base | Strains/genus with the most | Homology |
|---|---|---|---|
| Strain | (base pair) | similar sequence in Genbank | (%) |
| DH10 | 1404 | NR_108681.1 | 99.72% |
| | | Tessaracoccus oleiagriola | |

The strain DH10 of the disclosure can adopt the following preservation methods:

(1) short-term preservation, which specifically includes: the above strains are streaked on a slant medium, then cultured at 35° C. for 48 h, and stored at 4° C. for a short time.

(2) long-term preservation, which specifically includes: glycerol cryopreservation method is adopted, that is, several rings of bacteria are scraped from a fresh slant medium and transferred into a glycerol tube containing 1.5 ml of 30% sterilized glycerol for cryopreservation at −70° C.

Alternatively, long-term preservation is performed by adopting a method of skimmed milk cryopreservation, that is, several rings of bacteria are scraped from the fresh slant medium and transferred into a glycerol tube with sterilised skim milk for cryopreservation at −70° C.

The Tessaracoccus oleiagri strain DH10 screened by the disclosure has been preserved in China center for type culture collection (CCTCC) on Apr. 19, 2021, with the address of Wuhan University, Wuhan City, China, and the preservation number is CCTCC No: M 2021404.

### Embodiment 2 Decomposition of Montmorillonite by Strain DH10

1. Strain

the Tessaracoccus oleiagri strain DH10 screened in the embodiment 1.

2. Culture Medium

The composition of culture medium is: 10-50 grams per liter (g/L) of sucrose, 10-40 g/L of sodium acetate, 3-20 g/L of sodium lactate, 0.1-2 g/L of MgSO₄, 2-18 g/L of KCl, 5-9 g/L of MnSO₄, 5-10 g/L of CuSO₄, 5-12 g/L of ZnSO₄, 1-7 g/L of KH₂PO₄ and 2-10 g/L of montmorillonite. A range of pH value of the culture medium is 5.0-9.0. After the preparation of the culture medium, it is sterilized at 0.1 megapascals (MPa) for 30 minutes.

3. Fermentation Cultivation

Slant preserved strains were activated by streaking on the plate with an inoculating loop, and cultured at 35° C. for 20 hours. Subsequently, three rings of strains (strains on each inoculation ring contains more than 3 single colonies with obvious characteristics) are picked from the plate, then put into a shake flask (50 ml triangular flask, with a liquid volume of 30 ml), cultured at 35° C. for 8 hours at 180 revolutions per minute (r/min), and centrifuged and collected, so that bacteria are obtained. The obtained bacteria are washed with sterile water for 3 times, then resuspended, and transferred into a montmorillonite medium for anaerobic culture at 35° C.

4. Reduction of Fe(III) in Clay Minerals by Microorganisms

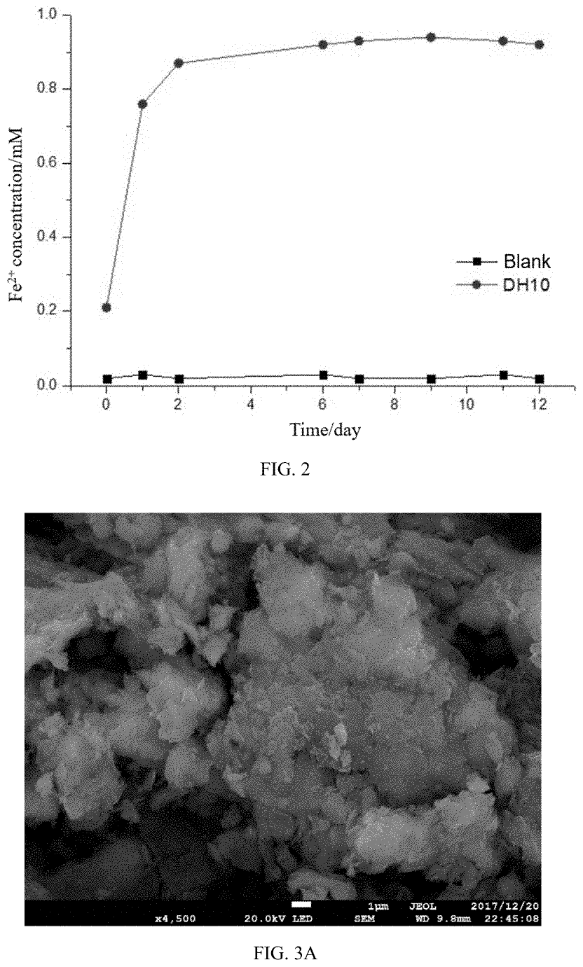

The phenanthroline spectrophotometry is used to measure Fe(II) content, and specific operation refers to a Chinese standard “determination of iron phenanthroline spectrophotometry” of HJ/T 345-2007. After the Tessaracoccus oleiagri strain DH10 is transferred, a total Fe content in the sample is determined according to the method in the standard. After culture, 5 ml of extract is taken at intervals, the Fe(II) content is calculated using a standard curve for determination of ferrous content, and a reduction efficiency of Fe(III) in the sample is calculated. After the Tessaracoccus oleiagri strain DH10 is inoculated, a change of Fe²⁺ concentration in montmorillonite with time compared with a blank control group is shown in FIG. 2. The results show that the Tessaracoccus oleiagri strain DH10 can achieve high-efficiency reduction of Fe(III) in montmorillonite, with a reduction efficiency of 41.9%.

5. Observation of the microscopic morphology of clay minerals before and after an action of microorganisms

Changes of in structure, morphology and mineralogy of the clay minerals before and after the action of the Tessaracoccus oleiagri strain DH10 are observed by scanning electron microscope (SEM).

A sample preparation method is as follows: (1) 1 ml of bacterial solution in the logarithmic phase is taken into a 1.5 ml eppendorf (EP) tube, centrifuged at 13400 r/min for 5 min at 4° C., and the supernatant is removed to obtain a first precipitate; (2) the first precipitate is washed with 1 ml of phosphate buffered saline (PBS) with pH 7.4, centrifuged at 13400 r/min for 5 min at 4° C., then the supernatant is removed, the operation is repeated for 3 times, thereby obtaining a target precipitate; (3) the target precipitate is placed on a clean slide glass and fixed with 2% paraformaldehyde-2.5% glutaraldehyde as a fixing solution for 1 hour to obtain fixed samples; (4) the fixed samples are dehydrated with different concentrations of ethanol, the concentrations of ethanol are 25%, 50%, 75%, 95% and 100%, and each concentration is dehydrated twice for 15-20 min each time, thereby obtaining dehydrated samples; (5) after dehydration, the dehydrated samples are put into a vacuum freeze dryer for 24 hours to obtain dried samples; (6) the dried samples are sprayed with gold in vacuum to thereby obtain samples for observation. The microscopic morphologies of montmorillonite before and after microbial action at different magnification are observed by scanning electron microscope.

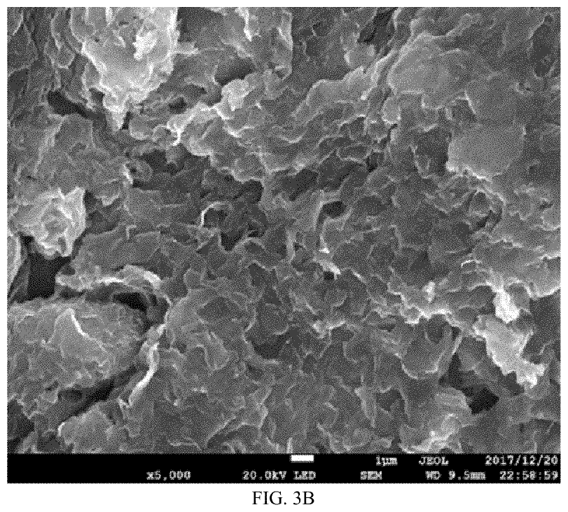

The observation results of scanning electron microscope are shown in FIG. 3A and FIG. 3B, where FIG. 3A shows an original sample of montmorillonite and FIG. 3B shows a sample of montmorillonite after 7 days of action of the strain DH10. The results show that the original montmorillonite samples are relatively compact solid particles gathered together, and the montmorillonite samples show a loose network structure after 7 days of the action of the Tessaracoccus oleiagri strain DH10, that is, the montmorillonite samples appear obvious dissolution phenomenon after the action of Tessaracoccus oleiagri strain DH10.

### Embodiment 3 Determination of Expansion Inhibition Rate

On the basis of the embodiment 2, referring to a method for measuring expansion inhibition rate of clay in standard clay stabilizer, expansion properties of montmorillonite minerals before and after the action of Fe(III)-reducing Tessaracoccus oleiagri strain DH10 are measured. The experimental steps are as follows: 0.1 gram (g) of dried montmorillonite sample of the control group is weighed and then placed into a 2.0 ml centrifuge tube, and a volume V0 of the sample before expansion in the centrifuge tube is recorded; then 1.5 ml of expansion medium solution (distilled water and kerosene as expansion medium solution respectively) are added and shaken uniformly to thereby a uniform sample; the uniform sample is placed at room temperature for 24 hours and then put into a centrifuge, centrifuged for 15 minutes at 5000 r/m, and a volume V1 of the sample after expansion is read. The above steps are repeated to measure the expansion volume of montmorillonite samples in the experimental group, and the volumes before and after expansion are recorded as V2 and V3 respectively. A formula for calculating the expansion inhibition rate of montmorillonite sample is as follows:

$$η = ( 1 - \frac{V 3 - V 2}{V 1 - V 0} ) \times 100 \% ; \qquad ( 1 )$$

where η is an expansion inhibition rate, %; V0 is the volume of dried montmorillonite sample in the control group, ml; V1 is the expansion volume of montmorillonite in the expansion medium of the control group, ml; V2 is a volume of dried montmorillonite after microbial action, ml; and V3 is an expansion volume of montmorillonite in expansion medium after microbial action, ml.

The experimental results show that the Tessaracoccus oleiagri strain DH10 has a good effect on inhibiting the hydration expansion of clay minerals, in which the inhibition expansion rate is 50.61% in water system, and that in kerosene system is 87.63%. That is, the Tessaracoccus oleiagri strain DH10 of the disclosure has a good effect of inhibiting clay expansion.

### Embodiment 4 Reduction of Reservoir Injection Pressure by Iron-Reducing Strain DH10

Firstly, the formation water is used to test the permeability of low permeability reservoir, and then the cell suspension of the strain DH10 is injected and cultured for 2 weeks. Then, the formation water is used again to test the permeability of the low-permeability reservoir, so as to evaluate the effect of dissimilatory iron-reducing strain DH10 on the injection pressure of the low-permeability reservoir. The specific operation steps are as follows:

1. the core is baked and weighed, then placed in the core holder, confining pressure is applied and vacuum is pumped;

2. formation water is injected with a flow rate of 0.50 ml/min and an injection volume of 15 pore volumes (PV), and pressure difference between inlet and outlet of the core holder during the stable period is record;

3. the strain DH10 cell suspension is injected at a flow rate of 0.30 ml/min and an injection volume of 2 PV, then the inlet and outlet are closed and cultured at 55° C. for 2 weeks; and

4. the inject formation water are subsequently injected with a flow rate of 0.50 ml/min and an injection volume of 15 PV, and the pressure difference between the inlet and outlet of the core holder during the stable period is record.

Core parameters for evaluation of reducing the injection pressure of low permeability reservoir by strain DH10 are shown in Table 2, and the measurement results are shown in Table 3.

TABLE 2
core parameters for evaluation of reducing reservoir
injection pressure by iron-reducing strain DH10

| | | | Cross section | | Dry | Wet | |
|---|---|---|---|---|---|---|---|
| Core | Length | Diameter | area | Volume | weight | weight | Porosity |
| number | (cm) | (cm) | (cm²⁾ | (cm³) | (g) | (g) | (%) |
| MD2 | 7.725 | 2.500 | 4.906 | 37.899 | 74.057 | 79.688 | 14.64% |

TABLE 3
evaluation results of reducing reservoir injection
pressure by iron-reducing strain DH10

| | | | | Formation water displacement | | |
|---|---|---|---|---|---|---|
| | | | | after DH10 action | | |
| | | Formation water | | | | Pressure |
| | | displacement | | | | difference |
| | Flow | Pressure | | Pressure | | reduction |
| Core | velocity | difference | Permeability | difference | Permeability | percentage |
| number | (mL/min) | (MPa) | (10⁻³um²) | (MPa) | (10⁻³um²) | (%) |
| MD2 | 0.500 | 0.305 | 2.398 | 0.239 | 3.060 | 21.64% |

The results show that for core MD2 with permeability less than 10 10⁻³ um², the differential pressure when using formation water displacement is 0.305 MPa; After using iron-reducing bacteria DH10, the differential pressure is reduced to 0.239 MPa and the injection differential pressure is reduced by 21.64%, indicating that iron-reducing bacteria DH10 can significantly reduce the injection pressure of low permeability reservoir.

### Embodiment 5 Improvement of Crude Oil Recovery Factor by Iron-Reducing Strain DH10

The effect of strain DH10 on improving crude oil recovery factor is evaluated by using a core simulation experiment. The specific steps are as follows:

1. a core pretreatment is performed and a displacement device is connected;

2. formation water is saturated with a flow rate of 0.50 ml/min and an injection volume of 15 PV;

3. crude oil is saturated with a flow rate of 0.50 ml/min, the core outlet is observed until there is no formation water, an original oil content of the core from the volume of water flowing is calculated, and aged at 55° C. for 72 hours;

4. the formation water is used for primary water displacement with a flow rate of 0.50 ml/min, the volume of oil produced is measured when the water content at the core outlet is 98%, which is the volume of oil produced by primary water displacement.

5. the control group is injected with formation water added with 15 millimoles per liter (mM) sodium acetate, and the experimental group is injected with DH10 cell suspension; flow rate 0.30 ml/min, injection volume 2 PV, then the core inlet and core outlet are close, and cultured at 55° C. for 2 weeks;

6. subsequently, the formation water is used for displacement until the water content at the core outlet is 100%, the flow rate is 0.50 ml/min, and the volume of produced oil is measured, which is the volume of oil produced by secondary water displacement.

The core parameters of iron-reducing strain DH10 for enhanced oil recovery factors evaluation are shown in Table 4, and the evaluation results are shown in Table 5.

TABLE 4
core parameters for enhanced oil recovery factors evaluation of iron-reducing strain DH10

| | | | Cross section | | Dry | Wet | | Pore | |
|---|---|---|---|---|---|---|---|---|---|
| Core | Length | Diameter | area | Volume | weight | weight | Porosity | volume | Permeability |
| number | (cm) | (cm) | (cm²⁾ | (cm³) | (g) | (g) | (%) | (cm³) | (10⁻³um²) |
| ER0 | 7.843 | 2.500 | 4.906 | 38.478 | 83.987 | 94.054 | 25.78% | 9.918 | 82.494 |
| (control | | | | | | | | | |
| group) | | | | | | | | | |
| ER1 | 7.258 | 2.500 | 4.906 | 35.608 | 79.263 | 88.988 | 26.91% | 9.581 | 98.152 |
| ER2 | 7.375 | 2.500 | 4.906 | 36.182 | 74.385 | 82.894 | 23.17% | 8.383 | 69.814 |

TABLE 5
evaluation results of enhanced crude oil recovery factors by iron-reducing strain DH10

| | | | Primary water | | Secondary water displacement | | | |
|---|---|---|---|---|---|---|---|---|
| | | | displacement | | after DH10 action | | | |
| | | | | Primary | | Secondary | | |
| | | Original | Produced | water | | water | | |
| | Saturated | oil | oil | displacement | Produced | displacement | Enhanced oil | Average |
| Core | oil volume | saturation | volume | recovery | oil volume | recovery | recovery | value |
| number | (cm³) | (Decimal) | (cm³) | factor | (cm³) | factor | factor (%) | (%) |
| ER0 | 7.000 | 0.706 | 3.400 | 48.57% | 0.050 | 0.71% | / | / |
| (control | | | | | | | | |
| group) | | | | | | | | |
| ER1 | 6.900 | 0.720 | 3.200 | 46.38% | 0.450 | 6.52% | 5.81% | 5.32% |
| ER2 | 6.300 | 0.751 | 2.800 | 44.44% | 0.350 | 5.56% | 4.84% | |

The results show that using cores with a permeability of 70-100 10⁻³ um², the recovery factors of ER0, ER1 and ER2 are 48.57%, 46.38% and 44.44% respectively. After treatment with iron-reducing strain enrichment DH10, the recovery factors of secondary water displacement are 0.71%, 6.52% and 5.56% respectively. Compared with the control group ER0, the recovery factors of ER1 and ER2 in the experimental group are increased by 5.81% and 4.84% respectively, with an average of 5.32%.

In conclusion, the iron-reducing Tessaracoccus oleiagri strain DH10 is separated and screened by the disclosure, which can effectively decompose and corrode montmorillonite minerals and inhibit clay expansion. At the same time, core experiments show that the Tessaracoccus oleiagri strain DH10 can reduce the water injection pressure in oil development, apply it to crude oil recovery, and significantly improve the recovery factor of crude oil.

The above are only the illustrated specific embodiments of the disclosure, but the protection scope of the disclosure is not limited to this. Any change or replacement that can be easily thought of by any technician familiar with the technical field within the technical scope disclosed by the disclosure shall be included by the protection scope of the disclosure.

What is claimed is:

1. An iron-reducing Tessaracoccus oleiagri strain DH10, wherein a preservation number of the strain DH10 is CCTCC No: M 2021404.

2. A microbial agent, comprising:
the iron-reducing Tessaracoccus oleiagri strain DH10 according to claim 1.

3. The microbial agent according to claim 2, wherein the microbial agent is a solid preparation or a liquid preparation.

4. A biological expansion-shrinking bacteria agent, comprising:
a nutrient medium; and
the iron-reducing strain Tessaracoccus oleiagri DH10 according to claim 1.

5. The biological expansion-shrinking bacteria agent according to claim 4, wherein the nutrient medium is a lysogeny broth (LB) medium; or
wherein the nutrient medium is a culture medium with a range of pH value of 5.0-9.0 comprising:
10-50 grams per liter (g/L) of sucrose,
10-40 g/L of sodium acetate,
3-20 g/L sodium lactate,
0.1-2 g/L of magnesium sulfate (MgSO₄),
2-18 g/L of potassium chloride (KCl),
5-9 g/L of manganese sulfate (MnSO₄),
5-10 g/L of copper sulfate (CuSO₄),
5-12 g/L of zinc sulfate (ZnSO₄),
1-7 g/L of potassium dihydrogen phosphate (KH₂PO₄), and
2-10 g/L of montmorillonite.

6. The biological expansion-shrinking bacteria agent according to claim 4, wherein the biological expansion-shrinking bacteria agent is prepared by inoculating the iron-reducing Tessaracoccus oleiagri strain DH10 into the nutrient medium and then fermenting at pH 5-9.5 and temperature 20-60° C.

7. The microbial agent according to claim 2, wherein the microbial agent is applied to reduction of Fe(III).

8. The microbial agent according to claim 2, wherein the microbial agent is applied to expansion-inhibiting of clay.

9. The microbial agent according to claim 2, wherein the microbial agent is applied to reducing of reservoir injection pressure.

10. The microbial agent according to claim 2, wherein the microbial agent is applied to enhancement of crude oil recovery factor.
